(19) United States
(12) Patent Application Publication
(10) Pub. No.: US 2009/0227692 A1
(43) Pub. Date: Sep. 10, 2009

(54) Biomarkers for breast cancer

(75) Inventors: Jinong Li, Ellicott City, MD (US); Saraswati Sukumar, Columbia, MD (US); Daniel W. Chan, Clarksville, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/920,906

(22) PCT Filed: May 25, 2006
(86) PCT No.: PCT/US2006/020643

§ 371 Date: Feb. 5, 2009

Related U.S. Application Data
(60) Provisional application No. 60/685,459, filed on May 26, 2005.

(51) Int. Cl. A61K 45/00; G01N 33/536; G01N 33/68; C07K 14/47; G01N 33/543

(52) U.S. Cl. 514/789; 436/536; 436/86; 530/324; 436/518

(57) ABSTRACT

The present invention provides protein-based biomarkers and biomarker combinations that are useful in qualifying breast cancer status in a patient. In particular, the biomarkers of this invention are useful to classify a subject sample as breast cancer or non-breast cancer. The biomarkers can be detected by SELDI mass spectrometry.

### RELATED APPLICATIONS

This application claims the benefit of U.S. Provisional Application No. 60/685,459, filed May 26, 2005, the entire contents of which is expressly incorporated herein by reference.

### FIELD OF THE INVENTION

The invention relates generally to clinical diagnostics.

### BACKGROUND OF THE INVENTION

Breast cancer is the most commonly diagnosed cancer among women. Presymtomatic screening to detect early-stage breast cancer while it is still resectable with potential for cure can greatly reduce breast cancer-related mortality. Unfortunately, only 63% (1992-1999, US) of the breast cancers are localized at the time of diagnosis (Jemal, A. et al. (2004) CA Cancer J. Clin. 54:8-29). Small lesions are frequently missed and may not be visible, even by mammography, particularly in young women and women with dense breast tissue (Antman, K. and Shea, S. (1999) JAMA 281:1470-1472). Molecular markers that can potentially identify these small lesions that are invisible to imaging techniques will provide a real opportunity to treat a neoplasm before it invades tissue.

Breast cancer is highly heterogeneous. Most molecular based approaches that have been investigated for the early detection of breast cancer are targeted at specific factors, such as oncogenes, tumor suppressor genes, growth factors, tumor antigens, or other gene products. The inherent problem is that none of these factors alone can account for a large majority of the breast cancers and some are not specific to cancer or to breast tissues, so the sensitivity and specificity of such approaches is low. So far, no molecular biomarkers are recommended for the early detection of breast cancer (Smith, R. A. et al. (2004) CA Cancer J. Clin. 54:41-52).

The human mammary gland is composed of discrete ductal-alveolar systems that originate at the nipple and branch dichotomously through the surrounding stroma toward the chest wall. Most breast carcinomas (70-80%) are thought to arise from the epithelial cells lining the terminal ducts of these structures. The breast epithelium exfoliates cells as a renewal tissue and secretes into fluids within the luminal compartment of the gland. These fluids exit each breast through six to nine separate orifices at the nipple, and can be collected using either of two non-invasive procedures: nipple aspiration and ductal lavage. In nipple aspiration, a simple handheld suction cup is placed on the nipple and used to quickly obtain concentrated fluid droplets at nipple openings. These droplets were collected with capillary tubes. This technique is successful in most women (Sartorius, O. W. et al. (1977) J. Natl. Cancer Inst. 59:1073-1080) and the yield typically varies between several to one hundred micro liters (Klein, P. et al. (2001) Breast J. 7:378-387; Hsiung, R. et al. (2002) Cancer J. 8:303-310). As NAF comes only from the immediate vicinity of the nipple and the yield of it is unpredictable, a ductal lavage system has been devised. This method involves suction of the nipple in order to localize NAF-yielding duct(s). NAF-producing duct(s) can then be cannulated using a micro-catheter and lavaged with saline. DLF may provide a better source of cells and proteins released from the tumor since it represents washes from the entire length of the duct.

Compared to serum, breast fluids potentially offer a superior source of biomarkers for breast cancer since the proteins present are specifically from breast tissue. Therefore, it would be beneficial to screen in breast fluid of a large patient cohort for a multiple protein panel that can identify majority of the breast cancer cases.

### BRIEF SUMMARY OF THE INVENTION

The present invention is based, at least in part, on the discovery of biomarkers for breast cancer in nipple aspiration fluid (NAF) and ductal lavage fluid (DLF). Accordingly, the invention provides a method for qualifying breast cancer status in a subject comprising: (a) measuring at least one biomarker in a biological sample from the subject, wherein the at least one biomarker is selected from the group consisting of the biomarkers of Table 1; and (b) correlating the measurement with breast cancer status.

In one embodiment, the at least one biomarker is measured by capturing the biomarker on an adsorbent surface of a SELDI probe and detecting the captured biomarkers by laser desorption-ionization mass spectrometry. In another embodiment, the at least one biomarker is measured by immunoassay.

In one embodiment, the adsorbent is a IMAC-Cu²⁺ adsorbent. In another embodiment, the adsorbent is a biospecific adsorbent (e.g., a biospecific adsorbent comprising antibodies to defensin-1, α-defensin-2, α-defensin-3 C-terminal fragment 1 of α-1-antitrypsin inhibitor or C-terminal fragment 2 of α-1-antitrypsin inhibitor).

In one embodiment, the correlating is performed by a software classification algorithm.

In another embodiment, breast cancer status is selected from breast cancer and non-breast cancer. In another embodiment, breast cancer status is selected from stage I or II primary in situ breast cancer and non-breast cancer.

In another embodiment, if the measurement correlates with breast cancer, the method further comprises administering at least one treatment to the subject selected from the group consisting of surgery, radiation, and chemotherapy.

In one embodiment, the at least one biomarker is α-defensin (e.g., α-defensin is α-defensin-1, α-defensin-2, or α-defensin-3). In another embodiment, the biomarker is a C-terminal fragment of α-1-antitrypsin inhibitor (e.g., C-terminal fragment 1 of α-1-antitrypsin inhibitor or C-terminal fragment 2 of α-1-antitrypsin inhibitor). In one embodiment, at least two biomarkers are measured. In another embodiment, at least three biomarkers are measured. In another embodiment, the method further comprises measuring at least one biomarker that is capable of differentiating between breast cancer and non-cancer, and wherein the biomarker is not α-defensin-1, α-defensin-2, α-defensin-3, BF-4, C-terminal fragment 1 of α-1-antitrypsin inhibitor or C-terminal fragment 2 of α-1-antitrypsin inhibitor.

In another embodiment, the invention provides a method for determining the course of breast cancer comprising: (a) measuring, at a first time, at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a biological sample from the subject; (b) measuring, at a second time, the at least one biomarker in a biological sample from the subject; and comparing the first measurement and the second measurement; wherein the comparative measurements determine the course of the breast cancer. In another embodiment, the method further comprises: (d) measuring the at least one biomarker after subject management and correlating the measurement with disease progression.

In another embodiment, the method comprises measuring at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a sample from a subject.

In another embodiment, the invention provides a composition comprising a purified biomarker, wherein the biomarker is selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor.

In another embodiment, the invention provides a composition comprising a biospecific capture reagent that specifically binds to a biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor.

In another embodiment, the invention provides a composition comprising a biospecific capture reagent bound to a biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor.

In another embodiment, the invention provides a kit comprising: (a) a solid support comprising at least one capture reagent attached thereto, wherein the capture reagent binds at least one biomarker selected from the group consisting of α-defensin-1, α-defensin-2, and α-defensin-3; and (b) instructions for using the solid support to detect the at least one biomarker. In one embodiment, the solid support comprising a capture reagent is a SELDI probe. In one embodiment, the capture reagent is an IMAC-Cu²⁺ adsorbent. In another embodiment, the kit further comprises: (c) a container containing at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor. In another embodiment, the kit further comprises: (c) a one Kd-cutoff dialysis reagent.

In another embodiment, the invention provides a kit comprising: (a) a solid support comprising at least one capture reagent attached thereto, wherein the capture reagents bind at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor; and (b) a container containing at least one of the biomarkers. In one embodiment, the solid support comprising a capture reagent is a SELDI probe. In one embodiment, the capture reagent is an IMAC-Cu²⁺ adsorbent.

In another embodiment, the invention provides a software product comprising: (a) code that accesses data attributed to a sample, the data comprising measurement of at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor; and (b) code that executes a classification algorithm that classifies the breast cancer status of the sample as a function of the measurement.

In another embodiment, the invention provides a method comprising detecting at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor by mass spectrometry or immunoassay.

In another embodiment, the invention provides a method comprising communicating to a subject a diagnosis relating to breast cancer status determined from the correlation of at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a sample from the subject. In one embodiment, the diagnosis is communicated to the subject via a computer-generated medium.

In another embodiment, the invention provides a method for identifying a compound that interacts with a biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor, wherein said method comprises: (a) contacting the biomarker with a test compound; and (b) determining whether the test compound interacts with the biomarker.

In another embodiment, the invention provides a method for modulating the concentration of a biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a cell, wherein said method comprises contacting said cell with a compound, wherein said compound modulates the expression or post-translational processing of the biomarker.

In another embodiment, the invention provides a method of treating a condition (e.g., breast cancer) in a subject, wherein said method comprises administering to a subject a therapeutically effective amount of a compound, wherein said compound modulates the expression or post-translational processing of a biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor.

### BRIEF DESCRIPTION OF THE DRAWINGS

FIG. 1 depicts a pseudo-gel view of protein profiles of nipple aspiration fluid from five patients with primary invasive cancer (C6, C11, C14, C16, and C26) and five normal controls (N4, N15, N32, N33, and N36). Mass spectra was obtained on IMAC-Cu chip arrays using 1 μg of total protein. General protein expression profiles of NAF from different individuals are variable, whereas mass spectra of triplicate analysis of the same specimen are highly reproducible.

FIG. 2 depicts biomarker training and testing. Training sample: NAF (Panel A and B, representing the two subgroups recognized by unsupervised cluster analysis). Testing sample: pooled DLF collected (Panel C). The selected biomarkers are indicated by arrow heads. BF1-3 appear as a cluster of three peaks (M/Z 3375, 3447, and 3490) and were elevated in C6 and C14 and selected as the most effective discriminators in Panel A. BF4 (M/Z) 4079 was elevated in C1 and C16, and BF5 (M/Z 4680) was elevated in C26; these two markers collectively can discriminate cancer versus non-cancer specimens in Panel B. Elevation of BF1-3 and BF5 in cancer was validated in testing data.

FIG. 3 demonstrates antibody capture of BF1-3 using a monoclonal antibody against HNP1-3. The antibody was amino-linked to AminoLink beads and incubated with two NAF specimens with high BF1-3 peaks (NAF-C6 and NAF-C14). Shown are original mass spectra of NAF-C6 (A) and C14 (C); along with mass spectra of the captured proteins from same two specimens (B and D, respectively).

FIG. 4 depicts a quantitative analysis of HNP1-3 by ELISA. High peak amplitude of BF1-3 by SELDI analysis (top) correlates with high level of HNP1-3 measured by ELISA (bottom).

FIG. 5 depicts the level of HNP1-3 (measured by ELISA) in 42 DLF samples from 13 women at high risk for breast cancer (A); and corresponding protein concentration in each sample (B). Persistent elevation on HNP1-3 is observed in Patient 11 (5 out of her 6 samples from two time point) and low expression of HNP is not due to lack of protein in sample.

FIG. 6 depicts data showing differentially expressed BC1-3 in subgroup A; BC4 and 5 in subgroup B. BC1-3 was later identified as human neutrophil peptide 1-3 (HNP1-3). Split of Group A and B was based on similarity between mass spectra; recognized by unsupervised cluster analysis. All NAF specimens were obtained from M. D. Anderson Cancer Center. Elevation of HNP1-3 and BC5 was also observed in a pair of pooled DLF specimens obtained from Johns Hopkins University.

FIG. 7 demonstrates elevated expression of BC5 (arrow head) was observed in cancer/disease breast of 4 subjects with invasive cancer (4/25); 1 subject with DCIS (1/3), and 1 subject with ADH (1/3). None of the contra-lateral control breasts showed positive for BC5, including breasts of all control subjects (data not shown). Reproducibility of mass spectra was shown using data from DCIS and ADH subjects. Mass spectrum of C26 from previous data was included for comparison purpose

FIG. 8 depicts the level of HNP1-3 (ng/μg) as measured by ELISA. A, NAF samples from the cancer breasts of 5 cancer subjects (c) and breasts of 5 healthy controls (n) were obtained from M. D. Anderson Cancer Center. Elevation of HNP1-3 was observed in 2 of 5 cancer cases, at level of 70-80 ng/μg; B, Previous data, DLF obtained from Northwestern University. Bilateral ductal lavage was performed every 6-12 months on women at high risk of breast cancer (5-year Gail risk>1.6 or history of previous lobular carcinoma). Persistent elevation of HNP1-3 was observed in subject 11; C, NAF. 1, Invasive subject-control breast; 2, Invasive subject-cancer breast, 3. DCIS-control breast, 4, DCIS-cancer breast, 5, ADH-control breast, 6, ADH-disease breast; 7, Control subject-low risk (No first degree relative family history and <2 previous biopsies); 8, Control subject-high risk (first degree relative family history or >=2 previous biopsies). D, Current data, DLF collected at Northwestern University. Base line expression of HNP1-3 was measured in 149 bilateral ductal lavage specimens from 58 high risk subjects (5-year Gail risk>1.6 or history of previous lobular carcinoma).

FIGS. 9A-B depicts mass spectrum of tryptic digests of BF-5. A: Mass Spectrum of a tryptic digest of BF-5. B: Mascot labeling of fragmentation of masses for peak at 1842 of tryptic digest from A.

FIGS. 10A-B depicts mass spectrum of BF-5. A: Mass Spectrum of BF-5. B: Predicted mass spectrum of BF-5-2.

### DETAILED DESCRIPTION OF THE INVENTION

### 1. Introduction

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

### 2. Biomarkers for Breast Cancer

2.1. Biomarkers

This invention provides polypeptide-based biomarkers that are differentially present in subjects having breast cancer, in particular, breast cancer versus normal (non-breast cancer). The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The biomarkers were discovered using SELDI technology employing ProteinChip arrays from Ciphergen Biosystems, Inc. (Fremont, Calif.) (“Ciphergen”). Samples were collected by nipple aspiration or ductal lavage from subjects diagnosed with breast cancer (biopsy-proven state I or II unilateral primary invasive breast cancer) and subjects diagnosed as normal. The samples were applied to SELDI biochips, and spectra of polypeptides in the samples were generated by time-of-flight mass spectrometry on a Ciphergen PBSII mass spectrometer. ProteinChip Software 3.0 (Ciphergen) was used to collect and evaluate the raw spectra. Qualified mass peaks (visual examination) with signal/noise>5 were manually selected, and the peak intensities were normalized to the total ion current of the selected mass region (in this case, between 3 kDa and 135 kDa. The peak intensities identified in replicate analysis were averaged and then log transformed for subsequence analysis. This method is described in more detail in the Example Section.

The biomarkers thus discovered are presented in Table 1. The “ProteinChip assay” column refers to chromatographic fraction in which the biomarker is found, the type of biochip to which the biomarker binds and the wash conditions, as per the Example.

TABLE 1

| Marker (with | | Up or down | |
|---|---|---|---|
| alternate | SEQ | regulated in | |
| names) | ID NO: | breast cancer | ProteinChip ® assay |
| α-defensin-2 | 2 | Up | IMAC-Cu, wash with PBS |
| (BF-1; HNP-2) | | | |
| M3375 | | | |
| α-defensin-1 | 1 | Up | IMAC-Cu, wash with PBS |
| (BF-2; HNP-1) | | | |
| M3447 | | | |
| α-defensin-3 | 3 | Up | MAC-Cu, wash with PBS |
| (BF-3; HNP-3) | | | |
| M3490 | | | |
| BF-4 | N/A | Up | IMAC-Cu, wash with PBS |
| M4079 | | | |
| C-terminal | 4 | Up | IMAC-Cu, wash with PBS |
| fragment 1 of α- | | | |
| 1-antitrypsin | | | |
| inhibitor (AAT; | | | |
| BF-5-1) | | | |
| M4680 | | | |
| C-terminal | 5 | Up | IMAC-Cu, wash with PBS |
| fragment 2 of α- | | | |
| 1-antitrypsin | | | |
| inhibitor (AAT; | | | |
| BF-5-2) | | | |
| M4680 | | | |

The biomarkers of this invention are characterized by their mass-to-charge ratio as determined by mass spectrometry. The mass-to-charge ratio of each biomarker is provided in Table 1 after the “M.” Thus, for example, M3447 has a measured mass-to-charge ratio of 3447. The mass-to-charge ratios were determined from mass spectra generated on a Ciphergen Biosystems, Inc. PBS-II ProteinChip Reader mass spectrometer. This instrument has a mass accuracy of about +/−0.15 percent. Additionally, the instrument has a mass resolution of about 400 to 1000 m/dm, where m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSII, taking the maximum and minimum mass-to-charge-ratio in the cluster, and dividing by two. Accordingly, the masses provided reflect these specifications.

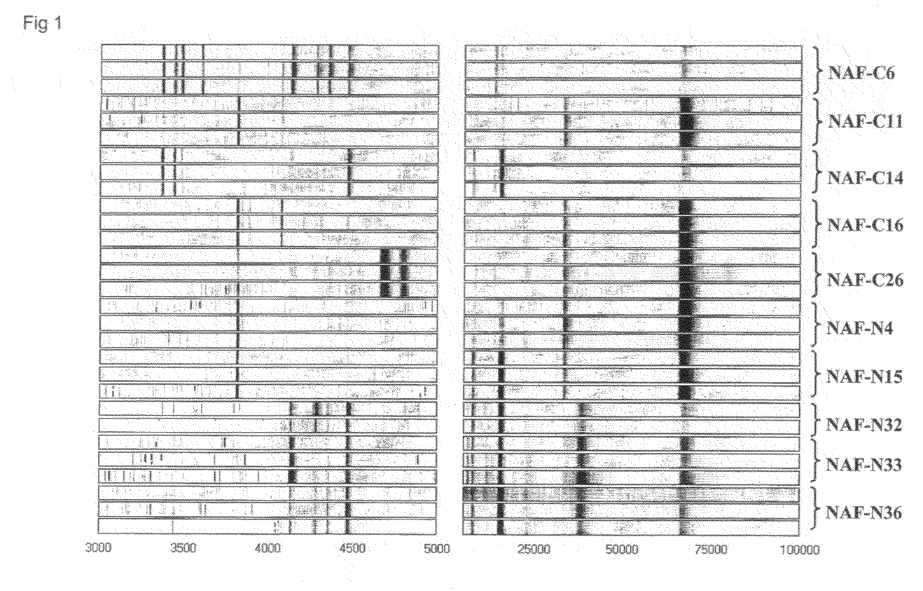

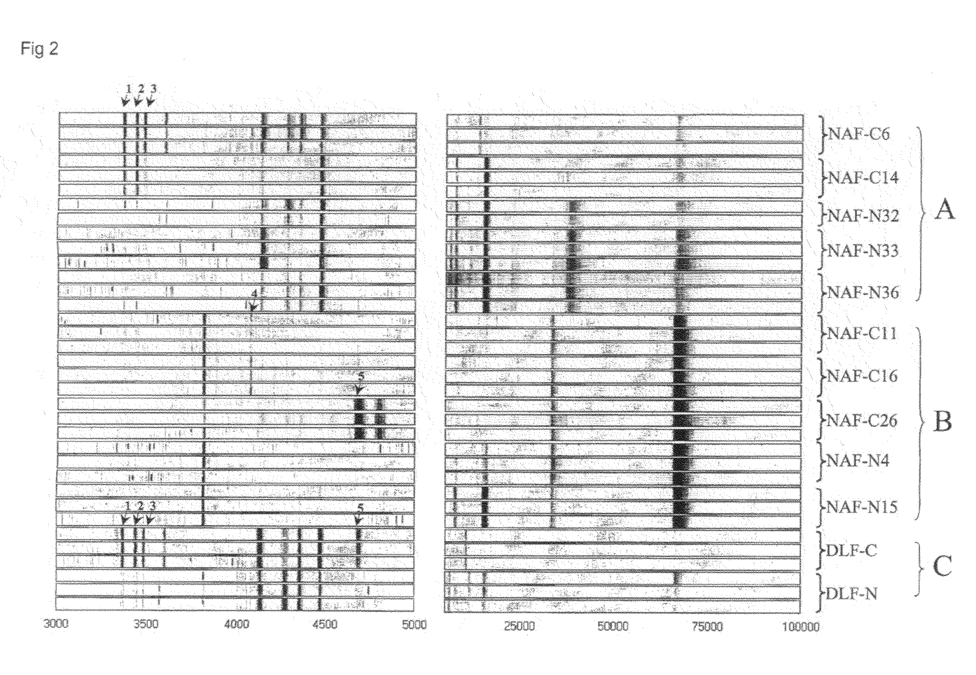

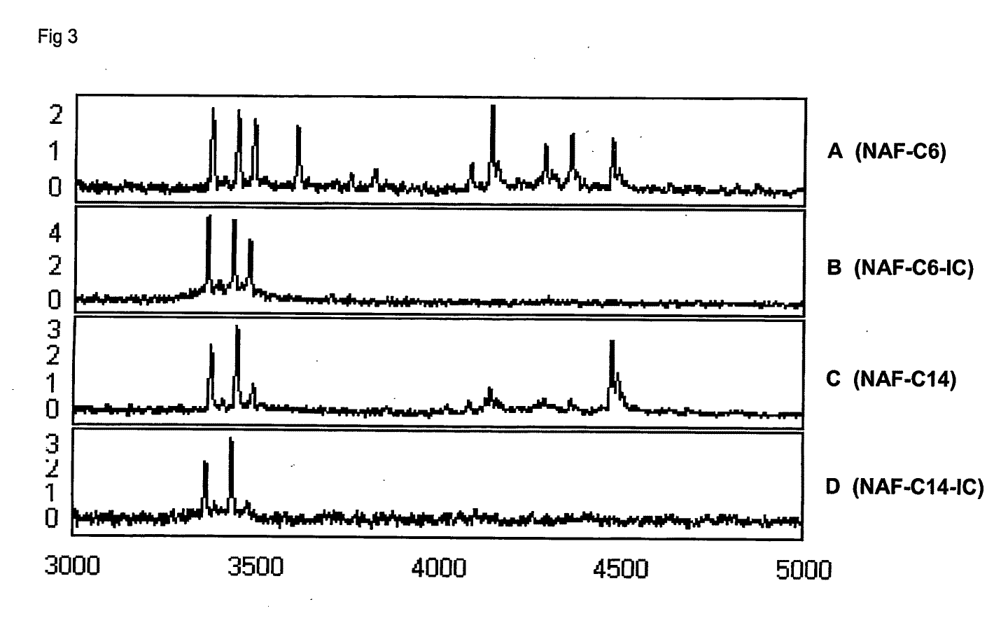

The biomarkers of this invention are further characterized by the shape of their spectral peak in time-of-flight mass spectrometry. Mass spectra showing peaks representing the biomarkers are presented in FIG. 3. Pseudo-gel views showing bands representing the biomarkers are presented in FIG. 1 and FIG. 2.

The biomarkers of this invention are further characterized by their binding properties on chromatographic surfaces. The biomarkers bind to metal affinity capture chip arrays (e.g., the Ciphergen® IMAC-Cu chip) after washing with phosphate-buffered saline (PBS).

The identities of certain of the biomarkers of this invention, BF-1, BF-2, BF-3, BF-5-1 and BF-5-2 have been determined, as indicated in Table 1. The method by which this determination was made is described in the Examples Section. For biomarkers whose identity has been determined, the presence of the biomarker can be determined by other methods known in the art.

Specifically, BF-1, 2, and 3 were identified as human neutrophil peptides 2, 1, and 3 (HNP-2, 1, and 3), respectively, which are also known as α-defensins-2, 1, and 3, respectively. HNP-1, 2, and 3 have been characterized as peptide antibiotics made principally by human neutrophils, even though some tumors may also produce α-defensin with the same capabilities. Recent studies have identified diverse function activities of HNP-1, 2, and 3, including tumor cell proliferation in renal cell carinoma (Muller, C. A. et al. (2002) Am. J. Pathol. 160:1311-1324). HNP-1, 2, and 3 are also known as α-defensins-1, 2, and 3, respectively (see U.S. Patent Application Publication No. US2004/0091498, incorporated herein by this reference). The amino acid sequences of human α-defensin-1 is ACYCRIPACIAGERRYGTCIYQGRLWAFCC (set forth as SEQ ID NO:1). The amino acid sequence of human α-defensin-2 is CYCRIPACIAGERRYGTCIYQGRLWAFCC (set forth as SEQ ID NO:2). The amino acid sequence of human α-defensin-3 is DCYCRIPACIAGERRYGTCIYQGRLWAFCC (set forth as SEQ ID NO:3).

Also, BF-5-1 was identified as a C-terminal fragment of α-1-antitrypsin inhibitor (AAT). The amino acid sequences of 13F-5-1 is LEAIPMSIPPEVK FNKPFVFLMIDQNTK SPLFMGKVVNPTQK (set forth as SEQ ID NO:4). BF-5-2 was identified as the C-terminal fragment of α-1-antitrypsin inhibitor (AAT). The amino acid sequences of BF-5-2 is EAIPMSIPPEVKFNKPFVFLMIDQNTK SPLFMGKVVNPTQK (set forth as SEQ ID NO:5).

Accordingly, exemplary biomarkers that are useful in the methods of the present invention are α-defensins. α-defensins are a family of proteins comprising about 94 amino acid residues. Specifically, α-defensin 3 is a 94 amino acid protein which is the expression product of a gene having GenBank Accession No. NP_{—}005208. SEQ ID NO:3 is a C-terminal fragment of α-defensin 3. α-defensin 1 is a 94 amino acid protein which is the expression product of a gene having GenBank Accession No.: NP_{—}004075 α-defensins are recognized by antibodies from AbCam (Catalog Number ab12757) (Cambridge, Mass.) Specific α-defensin biomarkers are presented in Table 1 as SEQ ID NOs: 1-3.

Further biomarkers that are useful in the methods of the present invention are C-terminal fragments of α-1-antitrypsin inhibitor (AAT). α-1-antitrypsin inhibitor is protein comprising about 418 amino acid residues and is the expression product of a gene having GenBank Accession No.: KO 396. Exemplary markers of the invention include C-terminal fragments of α-1-antitrypsin inhibitor, e.g., fragments that are approximately 30, 35, 40, 45 or 50 amino acid residues in length. α-1-antitrypsin inhibitor are recognized by antibodies from AbCam (Catalog Number ab9399) (Cambridge, Mass.). Specific α-1-antitrypsin inhibitor biomarkers are presented in Table 1 as SEQ ID NOs: 4 and 5.

Because the biomarkers of this invention are characterized by mass-to-charge ratio, binding properties and spectral shape, they can be detected by mass spectrometry without knowing their specific identity. However, if desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the biomarkers is nipple aspiration fluid (NAF) or ductal lavage fluid (DLF). However, in other embodiments, the biomarkers can be detected in blood, serum, plasma, or urine.

The biomarkers of this invention are biomolecules. Accordingly, this invention provides these biomolecules in isolated form. The biomarkers can be isolated from biological fluids, such as NAF or DLF. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

### 3. Biomarkers and Different Forms of a Protein

Proteins frequently exist in a sample in a plurality of different forms. These forms can result from either or both of pre- and post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation.

When detecting or measuring a protein in a sample, the ability to differentiate between different forms of a protein depends upon the nature of the difference and the method used to detect or measure. For example, an immunoassay using a monoclonal antibody will detect all forms of a protein containing the eptiope and will not distinguish between them. However, a sandwich immunoassay that uses two antibodies directed against different epitopes on a protein will detect all forms of the protein that contain both epitopes and will not detect those forms that contain only one of the epitopes.

In diagnostic assays, the inability to distinguish different forms of a protein has little impact when the forms detected by the particular method used are equally good biomarkers as any particular form. However, when a particular form (or a subset of particular forms) of a protein is a better biomarker than the collection of different forms detected together by a particular method, the power of the assay may suffer. In this case, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. First, a biospecific capture reagent (e.g., an antibody, aptamer or Antibody that recognizes the biomarker and other forms of it) is used to capture the biomarker of interest. Preferably, the biospecific capture reagent is bound to a solid phase, such as a bead, a plate, a membrane or an array. After unbound materials are washed away, the captured analytes are detected and/or measured by mass spectrometry. (This method also will also result in the capture of protein interactors that are bound to the proteins or that are otherwise recognized by antibodies and that, themselves, can be biomarkers.) Various forms of mass spectrometry are useful for detecting the protein forms, including laser desorption approaches, such as traditional MALDI or SELDI, and electrospray ionization.

Thus, when reference is made herein to detecting a particular protein or to measuring the amount of a particular protein, it means detecting and measuring the protein with or without resolving various forms of protein. For example, the step of “measuring α-defensin” includes measuring α-defensin by means that do not differentiate between various forms of the protein in a sample (e.g., certain immunoassays do not differentiate α-defensin-1, α-defensin-2 and α-defensin-3) as well as by means that differentiate some forms from other forms or that measure a specific form of the protein (e.g., any and/or all of α-defensin-1, α-defensin-2 and α-defensin-3, individually or in combination). In contrast, when it is desired to measure a particular form or forms of a protein, the particular form or forms are specified. For example, “measuring α-defensin-1” means measuring α-defensin-1 in a way that distinguishes it from other forms of α-defensin, e.g., α-defensin-2 and α-defensin-3. Similarly, reference to “measuring the C-terminal fragment of α-1-antitrypsin inhibitor” includes measuring any and/or all forms of C-terminal fragments of α-1-antitrypsin inhibitor, e.g., C-terminal fragment 1 of α-1-antitrypsin inhibitor and/or C-terminal fragment 2 of α-1-antitrypsin inhibitor, found in a subject test sample, individually or in combination.

### 4. Detection of Biomarkers for Breast Cancer

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

4.1. Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI

4.1.1. SELDI

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI also has been called is called “affinity capture mass spectrometry” or “Surface-Enhanced Affinity Capture” (“SEAC”). This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50′ (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Publication No. US 2003-0218130 A1 (Boschetti et al., “Biochips With Surfaces Coated With Polysaccharide-Based Hydrogels,” Apr. 14, 2003) and U.S. Patent Publication No. U.S. 2005-059086 A1 (Huang et al., “Photocrosslinked Hydrogel Blend Surface Coatings,” Mar. 17, 2005).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI chip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

4.1.2. SEND

Another method of laser desorption mass spectrometry is called Surface-Enhanced Neat Desorption (“SEND”). SEND involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylrnethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of laser desorption mass spectrometry in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

4.1.3. SEPAR

Another version of LDI is called Surface-Enhanced Photolabile Attachment and Release (“SEPAR”). SEPAR involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

4.1.4. MALDI

MALDI is a traditional method of laser desorption/ionization used to analyte biomolecules such as proteins and nucleic acids. In one MALDI method, the sample is mixed with matrix and deposited directly on a MALDI chip. However, the complexity of biological samples such as serum or urine make this method less than optimal without prior fractionation of the sample. Accordingly, in certain embodiments with biomarkers are preferably first captured with biospecific (e.g., an antibody) or chromatographic materials coupled to a solid support such as a resin (e.g., in a spin column). Specific affinity materials that bind the biomarkers of this invention are described above. After purification on the affinity material, the biomarkers are eluted and then detected by MALDI.

4.1.5. Other Forms of Ionization in Mass Spectrometry

In another method, the biomarkers are detected by LC-MS or LC-LC-MS. This involves resolving the proteins in a sample by one or two passes through liquid chromatography, followed by mass spectrometry analysis, typically electrospray ionization.

4.1.6. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

4.1.7. General Protocol for SELDI Detection of Biomarkers for Breast Cancer

A preferred protocol for the detection of the biomarkers of this invention is as follows. The biological sample to be tested, e.g., NAF, may be applied directly to the biochip without any additional manipulation. The sample, e.g., DLF, may be applied directly to the biochip after lyophilization and dialysis to remove excess saline. The samples may also be fractionated, e.g., by size-exclusion chromatography, anion- or cation-exchange chromatography, or other fractionation methods.

The sample to be tested is then contacted with an affinity capture probe comprising an IMAC adsorbent (preferably an IMAC30-Cu ProteinChip array (Ciphergen Biosystems, Inc.)), again as indicated in Table 1. The probe is washed with a buffer that will retain the biomarker while washing away unbound molecules. A suitable wash for each biomarker is the buffer identified in Table 1. The biomarkers are detected by laser desorption/ionization mass spectrometry.

Alternatively, if antibodies that recognize the biomarker are available, for example in the case of α-defensins-1-3, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

4.2. Detection by Immunoassay

In another embodiment, the biomarkers of this invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### 5. Determination of Subject Breast Cancer Status

5.1. Single Markers

The biomarkers of the invention can be used in diagnostic tests to assess breast cancer status in a subject, e.g., to diagnose breast cancer. The phrase “breast cancer status” includes any distinguishable manifestation of the disease, including non-disease. For example, disease status includes, without limitation, the presence or absence of disease (e.g., breast cancer v. non-breast cancer), the risk of developing disease, the stage of the disease, the progress of disease (e.g., progress of disease or remission of disease over time) and the effectiveness or response to treatment of disease. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives who test as positive. Negative predictive value is the percentage of actual negatives that test as negative.

The biomarkers of this invention show a statistical difference in different breast cancer statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98% and about 100%.

Each biomarker listed in Table 1 is differentially present in breast cancer, and, therefore, each is individually useful in aiding in the determination of breast cancer status. The method involves, first, measuring the selected biomarker in a subject sample using the methods described herein, e.g., capture on a SELDI biochip followed by detection by mass spectrometry and, second, comparing the measurement with a diagnostic amount or cut-off that distinguishes a positive breast cancer status from a negative breast cancer status. The diagnostic amount represents a measured amount of a biomarker above which or below which a subject is classified as having a particular breast cancer status. For example, if the biomarker is up-regulated compared to normal during breast cancer, then a measured amount above the diagnostic cutoff provides a diagnosis of breast cancer. Alternatively, if the biomarker is down-regulated during breast cancer, then a measured amount below the diagnostic cutoff provides a diagnosis of breast cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different breast cancer statuses, as was done here, and drawing the cut-off to suit the diagnostician's desired levels of specificity and sensitivity.

5.2. Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. A combination of at least two biomarkers is sometimes referred to as a “biomarker profile” or “biomarker fingerprint.” Under certain circumstances (e.g., when the biomarkers are detected using a biospecific reagent such as an antibody), α-defensins-1, 2, or 3 are detected as a single biomarker, referred to herein as “α-defensin”. Under other circumstances, C-terminal fragment 1 of α-1-antitrypsin inhibitor and C-terminal fragment 2 of α-1-antitrypsin inhibitor are detected as a single biomarker, referred to herein as “C-terminal fragments of α-1-antitrypsin inhibitor”.

The peak intensity data obtained from 10 NAF specimens (see Examples below) as the training data for selection of candidate biomarkers. Due to the individual viability of the mass spectra obtained (visual inspection), an unsupervised cluster analysis (MATLAB) was first performed. Two clusters were observed, one consists of specimens C6, C14, N32, N33 and N36 (Group A). The other consists of specimens C11, C16, C26, N4 and N15 (Group B). A subsequent supervised cluster analysis was then performed within each subgroup using ProPeak (3Z Informatics, Charleston, S.C.), and biomarkers that can effectively separate the cancer and non-cancer data were selected. ProPeak implements the linear version of the Unified Maximum Separability Analysis (UMSA) algorithm that was first reported for use in microarray data analysis.¹⁶ Application of ProPeak in SELDI protein array data analysis was described in detail previously.⁷ Briefly, each specimen was analyzed and projected as an individual point onto a three-dimensional component space, where location of each point was determined by linear regression derived composite index using peak intensity data. The rank of each peak represents its contribution towards the maximal separation of the cancer and non-cancer specimens. In this case, the peaks were visually inspected with high discriminatory power, and 5 peaks were selected (3 peaks in Group A, 2 peaks in Group B) that were elevated in cancer for further evaluation.

The potential biomarkers were tested on a pair of pooled DLF samples obtained from cancer and non-cancerous breast of patients with unilateral in situ ductal carcinoma (collected at Johns Hopkins Hospital). Equal amount of protein from 11 DLF samples of 9 cancerous breasts, 13 DLF of 7 non-cancerous breasts, respectively, were pooled to represent the cancer and non-cancer ducts. Protein profiles were generated in an independent experiment using the same chip protocol as described for the analysis of NAF.

5.3. Breast Cancer Status

Determining breast cancer status typically involves classifying an individual into one of two or more groups (statuses) based on the results of the diagnostic test. The diagnostic tests described herein can be used to classify between a number of different states.

5.3.1. Presence of Disease

In one embodiment, this invention provides methods for determining the presence or absence of breast cancer in a subject (status: breast cancer v. non-breast cancer). The presence or absence of breast cancer is determined by measuring the relevant biomarker or biomarkers and then either submitting them to a classification algorithm or comparing them with a reference amount and/or pattern of biomarkers that is associated with the particular risk level.

5.3.2. Determining Risk of Developing Disease

In one embodiment, this invention provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

5.3.3. Determining Stage of Disease

In one embodiment, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

5.3.4. Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers changes. Therefore, the trend of these markers, either increased or decreased over time toward diseased or non-diseased indicates the course of the disease. Accordingly, this method involves measuring one or more biomarkers in a subject at least two different time points, e.g., a first time and a second time, and comparing the change in amounts, if any. The course of disease is determined based on these comparisons.

5.4. Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers described herein is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

5.5. Subject Management

In certain embodiments of the methods of qualifying breast cancer status, the methods further comprise managing subject treatment based on the status. Such management includes the actions of the physician or clinician subsequent to determining breast cancer status. For example, if a physician makes a diagnosis of breast cancer, then a certain regime of treatment, such as prescription or administration of surgery, chemotherapy, and/or radiation might follow. Alternatively, a diagnosis of non-breast cancer or non-breast cancer might be followed with further testing to determine a specific disease that might the patient might be suffering from. Also, if the diagnostic test gives an inconclusive result on breast cancer status, further tests may be called for.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### 6. Generation of Classification Algorithms for Qualifying Breast Cancer Status

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for breast cancer. The classification algorithms, in turn, form the base for diagnostic tests by providing diagnostic values (e.g., cut-off points) for biomarkers used singly or in combination.

### 7. Compositions of Matter

In another aspect, this invention provides compositions of matter based on the biomarkers of this invention.

In one embodiment, this invention provides biomarkers of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, this invention provides biospecific capture reagents that specifically bind a biomarker of this invention, optionally in purified form. Preferably, a biospecific capture reagent is an antibody. In one embodiment, a biospecific capture reagent is an antibody that binds α-defensins-1, 2, and 3. Such an antibody is capable of specifically binding any of those three α-defensins, but cannot differentiate between them. In another embodiment, a biospecific capture reagent is an antibody that can differentiate between the three α-defensins. Such an antibody is likely to bind to the N-terminus of the biomarkers, where they differ in amino acid sequence.

In another embodiment, this invention provides biospecific capture reagents that specifically bind to C-terminal fragment 1 of α-1-antitrypsin inhibitor or C-terminal fragment 2 of α-1-antitrypsin inhibitor. Such an antibody is capable of specifically binding any of those two α-1-antitrypsin inhibitors, but cannot differentiate between them. In another embodiment, a biospecific capture reagent is an antibody that can differentiate between the two α-1-antitrypsin inhibitors. Such an antibody is likely to bind to the N-terminus of the biomarkers, where they differ in amino acid sequence.

In another embodiment, this invention provides a complex between a biomarker of this invention and biospecific capture reagent that specifically binds the biomarker. In other embodiments, the biospecific capture reagent is bound to a solid phase. For example, this invention contemplates a device comprising bead or chip derivatized with a biospecific capture reagent that binds to a biomarker of this invention and, also, the device in which a biomarker of this invention is bound to the biospecific capture reagent.

In another embodiment, this invention provides a device comprising a solid substrate to which is attached an adsorbent, e.g., a chromatographic adsorbent, to which is further bound a biomarker of this invention.

### 8. Kits for Detection of Biomarkers for Breast Cancer

In another aspect, the present invention provides kits for qualifying breast cancer status, which kits are used to detect biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### 9. Determining Therapeutic Efficacy of Pharmaceutical Drug

In another embodiment, this invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers of this invention changes toward a non-disease profile. For example, biomarkers α-defensins-1-3 are increased with disease. Therefore, one can follow the course of the amounts of these biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of the biomarkers at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. If a treatment is effective, then the biomarkers will trend toward normal, while if treatment is ineffective, the biomarkers will trend toward disease indications.

### 10. Use of Biomarkers for Breast Cancer in Screening Assays and Methods of Treating Breast Cancer

The methods of the present invention have other applications as well. For example, the biomarkers can be used to screen for compounds that modulate the expression of the biomarkers in vitro or in vivo, which compounds in turn may be useful in treating or preventing breast cancer in patients. In another example, the biomarkers can be used to monitor the response to treatments for breast cancer. In yet another example, the biomarkers can be used in heredity studies to determine if the subject is at risk for developing breast cancer.

Thus, for example, the kits of this invention could include a solid substrate having a metal affinity capture function, such as a protein biochip (e.g., a Ciphergen IMAC ProteinChip array, e.g., ProteinChip array), or a biospecific affinity capture function, such as a protein biochip comprising antibodies to α-defensins-1-3 or the C-terminal fragment of α-1-antitrypsin inhibitor, and a PBS buffer for washing the substrate, as well as instructions providing a protocol to measure the biomarkers of this invention on the chip and to use these measurements to diagnose breast cancer.

Compounds suitable for therapeutic testing may be screened initially by identifying compounds which interact with one or more biomarkers listed in Table 1. By way of example, screening might include recombinantly expressing a biomarker listed in Table 1, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers of Table 1 may also be measured. For example, the self-assembly of a multi-protein complex which includes one of the biomarkers of Table 1 may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table 1 may be administered to patients who are suffering from or are at risk of developing breast cancer or other cancer. For example, the administration of a test compound which increases the activity of a particular biomarker may decrease the risk of breast cancer in a patient if the activity of the particular biomarker in vivo prevents the accumulation of proteins for breast cancer. Conversely, the administration of a test compound which decreases the activity of a particular biomarker may decrease the risk of breast cancer in a patient if the increased activity of the biomarker is responsible, at least in part, for the onset of breast cancer.

In an additional aspect, the invention provides a method for identifying compounds useful for the treatment of disorders such as breast cancer which are associated with increased levels of modified forms of α-defensins-1-3 and/or the C-terminal fragment 1 and/or 2 of α-1-antitrypsin inhibitor. For example, in one embodiment, cell extracts or expression libraries may be screened for compounds which catalyze the cleavage of full-length α-defensins-1-3 or the C-terminal fragment 1 and/or 2 of α-1-antitrypsin inhibitor to form truncated forms of α-defensins-1-3 or the C-terminal fragment 1 and/or 2 of α-1-antitrypsin inhibitor, respectively. In one embodiment of such a screening assay, cleavage of α-defensins-1-3 or the C-terminal fragment 1 and/or 2 of α-1-antitrypsin inhibitor may be detected by attaching a fluorophore to α-defensins-1-3 or the C-terminal fragment 1 and/or 2 of α-1-antitrypsin inhibitor which remains quenched when α-defensins-1-3 or the C-terminal fragment 1 and/or 2 of α-1-antitrypsin inhibitor is uncleaved but which fluoresces when the protein is cleaved. Alternatively, a version of full-length C-defensins-1-3 or the C-terminal fragment 1 and/or 2 of α-1-antitrypsin inhibitor modified so as to render the amide bond between amino acids x and y uncleavable may be used to selectively bind or “trap” the cellular protease which cleaves full-length α-defensins-1-3 or the C-terminal fragment 1 and/or 2 of α-1-antitrypsin inhibitor at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the progression or likelihood of a disease, e.g., breast cancer, which is associated with the increased levels of truncated α-defensins-1-3 and/or the C-terminal fragment 1 and/or 2 of α-1-antitrypsin inhibitor. For example, after one or more proteins have been identified which cleave full-length α-defensins-1-3 or the C-terminal fragment 1 and/or 2 of α-1-antitrypsin inhibitor, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of α-defensins-1-3 and the C-terminal fragment 1 and/or 2 of α-1-antitrypsin inhibitor.

At the clinical level, screening a test compound includes obtaining samples from test subjects before and after the subjects have been exposed to a test compound. The levels in the samples of one or more of the biomarkers listed in Table 1 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table 1 may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table 1 may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with breast cancer, test compounds will be screened for their ability to slow or stop the progression of the disease.

### 11. Examples

### 11.1. Example 1

### Discovery of Biomarkers for Breast Cancer

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### 11.2. Example 2

### Materials and Methods

11.2.1. Multi-Center Study Design

To minimize the potential biases on patient selection as well as fluid collection procedures at each institution, and to maximize the applicability of the discovery, we recruited both nipple aspiration and ductal lavage fluid samples from three institutions, the University of Texas M. D. Anderson Cancer Center; the Sidney Kimmel Comprehensive Cancer Center at Johns Hopkins Hospital; and the Department of Surgery, Feinberg School of Medicine, Northwestern University. Sample collection at each site was approved by respective Institutional Review Board, and the current proteomic study is approved by the Institutional Review Board of Johns Hopkins University.

11.2.2. Patients—Nipple Aspiration Fluid (NAF)

NAF samples were obtained from the University of Texas M. D. Anderson Cancer Center. Patients who presented with biopsy-proven stage I or II unilateral primary invasive breast carcinoma were eligible for bilateral nipple aspiration. Patients were excluded from participation if they had previously undergone subareolar surgery that might have disrupted the terminal ductal system. Individuals were also eligible to participate if they were over 40 years of age and had no evidence of breast disease or cancer as evidenced by normal findings on physical examination and breast imaging. Ten fluid samples were available for this study, 5 were from the cancerous breast of breast cancer patients and the other 5 were from breast of healthy donors.

11.2.3. Collection Procedure—Nipple Aspiration Fluid (NAF)

Ductal fluid was collected by nipple aspiration using a handheld suction cup similar to nonpowered breast pumps used to express milk from lactating women. This device consists of a plastic cup connected to a section of polymer tubing. The tubing is attached to a standard syringe that is used to create a gentle vacuum. This device was originally used and described by Sartorius et al. (Sartorius, O. W. et al. (1977) J. Natl. Cancer Inst. 59:1073-1080) and was purchased for the collection of NAF from Product Health, Inc. (Menlo Park, Calif.).

Before aspiration was attempted, the nipple was cleansed with a small amount of Omniprep paste (D.O. Weaver and Co., Aurora, Colo.) to remove keratin plugs and then cleansed with an alcohol pad. A small amount of lotion was placed on the breast, and the breast was gently massaged from the chest wall towards the nipple for 1 minute. The suction cup was then placed over the nipple, and the plunger of the syringe was withdrawn to the 5-10 mL level until ductal fluid was visualized. The fluid droplets were collected into a 10 μL graduated micropipette (Drummond Scientific Co., Broomall, Pa.). Samples were obtained from both breasts, and the presence of NAF and volumes of NAF obtained were recorded for each patient and each breast.

Immediately after collection, the NAF-samples were rinsed into centrifuge tubes containing 500 μl sterile phosphate buffered saline supplemented with protease inhibitors AEBSF (4-[2-aminoethyl]-benzenesulfonylfluoride HCl; 0.2 mM), leupeptin (50 g/mL), aprotinin (2 g/mL), and dithiothreitol (DTT, 0.5 mM). The samples were then centrifuged at 1500 RPM for 10 minutes to remove insoluble materials and the supernatant was collected in 50 μl aliquots.

11.2.4. Patients—Ductal Lavage Fluids (DLF)

Two institutions contributed DLF samples to this study.

In a SPORE sponsored clinical trial at Johns Hopkins Hospital, women with Stage I or II biopsy-proven unilateral primary breast cancer are eligible for breast fluid collection prior to surgery. DLF from 1-3 ducts were collected individually from the patient's cancer bearing and the contralateral “normal” breast. At the time of the study, 42 specimens were available, 25 were from 9 breasts with cancer, 17 were from 7 contra-lateral breasts, and these specimens were from a total of 14 patients.

In a separate trial at Northwestern Hospital, women who were at increased risk because of a 5-year Gail risk estimate greater than 1.6%, one first-degree or two second-degree relatives with breast cancer, or history of LCIS, were recruited for tamoxifen treatment. These women undergo DL at entry, decide whether to take tamoxifen for breast cancer prevention, and undergo repeat DL 6-12 months later. 42 DLF specimens from 13 high risk women who chose not to take tamoxifen were available for this study.

11.2.5. Collection Procedure—Ductal Lavage Fluids (DLF)

Nipple aspiration is first performed to identify Fluid Yielding Duct (FYD). If nipple fluid is seen, the FYD is cannulated with a single lumen microcatheter and lavaged with normal saline. Anesthesia can be supplemented before cannulation with periareolar infiltration of approximately 5 mL of 1% lidocaine, and the ductal tree is often anesthetized by instilling 2 to 3 mL of 1% lidocaine through the nipple duct sphincter as soon as the tip of the catheter has been introduced into the duct orifice. Intermittent breast massage is performed after the instillation of approximately 2 mL of saline and this process is repeated four to five times, so that the total instilled volume is approximately 10 to 20 mL. The location of the fluid-yielding and cannulated ducts are recorded on an 8×8 grid, and photographed after inserting a prolene suture to facilitate recannulation at a later date in the Northwestern trial.

Immediately after collection, the samples were centrifuged at 1500 RPM for 10 minutes to remove insoluble materials and the supernatant was collected for subsequent proteomic analysis.

11.2.6. Sample Preparation for Proteomic Analysis

All samples were stored in −80° C. after collection, and frozen aliquots were shipped to Johns Hopkins on dry ice. No further processing was required for the NAF specimens received, whereas DLF was first lyophilized and then dialyzed overnight against phosphate buffered saline to remove excess saline using Tube-O-Dialyzer with 1 kDa molecular weight cut off (Upstate, N.Y.). Protein concentration in each fluid was measured using the BCA protein assay kit (Pierce, Rockford, Ill.).

11.2.7. SELDI Analysis

Various proteomic chip chemistries (hydrophobic, anionic, cationic, and metal affinity) were initially evaluated to determine which affinity chemistry provided the best profiles in terms of number and resolution of proteins. The Immobilized Metal Affinity Capture chip arrays (IMAC30) were selected. The active spots on IMAC30 contain nitrilotriacetic acid groups that chelate metal ions. Proteins bind to the chelated metal on IMAC30 arrays through histidine, tryptophan, cystein, or phosphorylated amino acids. Minimal amount of fluid protein required for each analysis, and binding and washing conditions were also tested for optimal protein presentation. Briefly, IMAC30 chip arrays were pretreated with CuSO4 using a 96-well format bioprocessor (hold twelve 8-spot chips) following the manufacture's instruction (Ciphergen Biosystems, CA). After incorporation of Cu2+ onto the chip surface, the bioprocessor was dissembled to release the chips. Various volumes (1-15 μl) of breast fluid samples containing 1 μg of protein were applied directly onto the pretreated spot and allowed to air-dry at room temperature. Allocation of specimens on protein chip arrays was randomized, including the triplicates of the same sample. The bioprocessor was reassembled after sample application and washed twice with 100 ml of PBS for 5 minutes followed by two quick rinses with 100 ml of dH2O to remove loosely bound materials. After air-drying, 0.5 ml of saturated sinapinic acid (SPA) prepared in 50% acetonitrile, 0.5% trifluoroacetic acid was applied twice to each spot as the energy absorbing molecules. Proteins bound to the chip surfaces were detected using a PBS-II ProteinChip Reader (Ciphergen Biosystems, CA). An automated analytical protocol was used to control the data acquisition process. Each spectrum was an average of 80 laser shots and externally calibrated against a mixture of known peptides or proteins. Molecular weight determination error was 0.05%.

11.2.8. Data Analysis

The data analysis process used in this study involved the following steps. (a) Peak detection. ProteinChip Software 3.0 (Ciphergen Biosystems, CA) was used to collect and evaluate the raw spectra. All mass spectra were compiled and baseline subtracted. Qualified mass peaks (visual examination) with signal/noise>5 were manually selected, and the peak intensities were normalized to the total ion current of the selected mass region. In this case, it was between 3 kDa and 135 kDa. The peak intensities at each M/Z identified in triplicate analysis were averaged and then log transformed for subsequent analysis. (b) Biomarker selection using training data. We have used the peak intensity data obtained from 10 NAF specimens as the training data for selection of candidate biomarkers. Due to the individual variability of the mass spectra obtained (visual inspection), an unsupervised cluster analysis (MATLAB) was first performed to recognize any potential patient subclasses based on general protein expression patterns. Two clusters were observed, one consists of specimens C6, C14, N32, N33 and N36 (Group A). The other consists of specimens C11, C16, C26, N4 and N15 (Group B). A supervised cluster analysis was then performed within each subgroup using ProPeak (3Z Informatics, Charleston, S.C.), and biomarkers that can effectively separate the cancer and non-cancer data were selected. ProPeak implements the linear version of the Unified Maximum Separability Analysis (UMSA) algorithm that was first reported for use in microarray data analysis (Zhang, Z. et al. In: Lin S M and Johnson K F (eds.), Methods of Microarray data analysis: papers from CAMDA '00. Boston: Kluwer Academic Publishers; 2001. p. 125-136). Application of ProPeak in SELDI protein array data analysis was described in detail previously (Li, J. et al. (2002) Clin. Chem. 48:1296-1304). Briefly, each specimen was analyzed and projected as an individual point onto a three-dimensional component space, where location of each point was determined by linear regression derived composite index using peak intensity data. The rank of each peak represents its contribution towards the maximal separation of the cancer and non-cancer specimens. In this case, we visually inspected the peaks with high discriminatory power, and selected 5 peaks (3 peaks in Group A, 2 peaks in Group B) that are elevated in cancer for further evaluation. (c) Biomarker validation using independent testing data. The validity of the potential biomarkers was tested on DLF specimens collected at the Johns Hopkins Hospital. Of the 42 1 ml DLF aliquots available for this study, 24 yielded more than 3 μg of protein needed for subsequent SELDI analysis. Equal amount of protein from 11 DLF of 9 cancerous breasts, 13 DLF of 7 non-cancerous breasts, respectively, were pooled to represent the cancer and non-cancer breasts. Protein profiles of the pooled specimens were generated in an independent experiment using the same chip protocol as described for the analysis of NAF.

11.2.9. SELDI-TOF-MS Immuno-Capture of BF1-3

Immuno-capture was performed using affinity purified rabbit antibody against a 16-aa peptide common to human HNP 1, 2 and 3 (Alpha diagnostics, San Antonio, Tex.). The antibody is linked to AminoLink beads using AminoLink Plus Immobilization Kit (Pierce, Rockford, Ill.) following manufacture's instructions. Two NAF specimens with high BF1-3 (C4 and C14) were used in the capture experiment and the captured peptides were analyzed on IMAC-Cu protein chip arrays as previously described.

11.2.10. Quantitative Measurement of HNP1-3 by ELISA

The level of HNP1-3 was measured using a sandwiched solid-phase enzyme-linked immunosorbent assay (ELISA). The kit was a product of HyCult Biotechnology, the Netherlands, distributed by Cell Sciences, Canton, Mass. Each sample was diluted (A pre-experiment was performed to determine the proper dilution factor for each sample), and measured in duplicates.

### 11.3. Example 3

### Results

11.3.1. Proteomic Profiling of Breast Fluids

Using our optimized chip protocol, we were able to obtain reproducible protein profiles using breast fluid samples containing 1 μg of total protein. FIG. 1 shows pseudo-gel view of protein profiles of nipple aspiration fluid from the cancerous breast of 5 patients with primary invasive cancer (C6, C11, C14, C16, C26) and from breasts of 5 normal controls (N4, N15, N32, N33, N36). General protein expression profiles of different individuals are variable, whereas mass spectra of triplicate analysis of the same specimen are highly reproducible. Ion signals of M/Z (Mass/Charge) less than 3000 are mainly noises of the matrix material, and the largest M/Z detected was at 135,000. We have manually selected 73 protein peaks (Signal/noise>5, M/Z of 3 K-135 K) for subsequent biomarker evaluation.

11.3.2. Biomarker Selection Using Training Data

The 10 NAF specimens was used as the training sample. First, we performed unsupervised cluster analysis to recognize any potential patient subclasses based on their protein expression data. Two clusters were formed, cluster A consisted of specimens C6, C14, N32, N33 and N36, and cluster B consisted of specimens C11, C16, C26, N4 and N15 (FIG. 2). These specimens were all collected on different days within a four month period in year 2001 under a standardized protocol, and no apparent differences on age, menopausal status were observed between the two subgroups.

To select biomarkers that are discriminatory within each subgroup, a subsequent supervised analysis was performed using ProPeak. The peaks were ranked based on their contribution towards the maximal separation of the cancer and non-cancer specimens within each group, and we selected 5 peaks (3 from Group A, 2 from Group B) with the highest discriminatory power for further evaluation. The M/Z values of the five selected peaks are 3375 (BF1), 3447 (BF2), 3490 (BF3), 4079 (BF4) and 4680 (BF5) (indicated by arrows in FIG. 2). BF1-3 appear as a cluster of three peaks and were elevated in C6 and C14, selected as the most effective discriminators in Group A. BF-4 was elevated in C11 and C16, and BF5 was elevated in C26. These two markers collectively can discriminate cancer versus non-cancer specimens in Group B. Collectively, a minimal of three peaks (BF1/2/3, BF4 and BF5) are needed to classify all 5 cancer cases correctly.

11.3.3. Biomarker Validation Using Independent Testing Data

The validities of BF1-5 were tested on a pair of pooled DLF specimens originated from samples collected at Johns Hopkins Hospital. Of the 42 1 ml DLF aliquots available, 24 yielded more than 3 μg of protein needed for SELDI analysis. These 24 samples included 11 DLF from 9 breasts with cancer, and 13 DLF from 7 contra-lateral cancer free breasts. Since only one duct of each cancerous breast harbors the tumor, and not all ducts were sampled, fluid yielding ducts from the cancerous breast may not necessarily contain the duct with the tumor. Initially, we planned to use cytology as the gold standard for identification of cancer ducts, but failed to do so due to lack of cells (<10 cells) in the majority of our samples (33/42). To increase the probability of getting a true positive and negative specimen representative of the cancer and non-cancer ducts, we created a pair of pooled DLF specimens by pooling equal amount of protein from all 11 ducts of the 9 cancerous breasts (DLF-C), and the 13 ducts of the 7 non-cancerous breasts (DLF-N), respectively. As shown in FIG. 2C, the general protein expression pattern of the pooled DLF samples resembles NAF of Group A, and elevations of BF1-3 and BF5 in cancer in comparison to the non-cancer controls were confirmed. It should be noted that elevation of BF1-3 and BF5 were previously observed in either Group A or B, the pooled DLF samples therefore present features of both subgroups. The Peak corresponding to BF4 was absent in the pooled DLF specimens, the validity of this marker therefore remain unverified.

BF1-3 was confirmed to be human neutrophil peptide 1-3 (HNP1-3). By searching through protein databases (National Center for Biotechnology Information and Swiss-Prot, both of which are available online), we found that BF1-3 of molecular weight 3375 (BF1), 3447 (BF2), and 3490 (BF3), correspond to the molecular masses of human neutrophil peptide 1-3 (HNP1-3). HNP1-3 are peptide antibiotics made principally by human neutrophils although some tumors might also produce HNP1-3 with the same capabilities. Besides their diverse functional activities in innate antimicrobial immunity, recent studies have also implicated its effect on tumor cell proliferation.

The identity of BF1-3 as HNP1-3 was verified by SELDI-TOF-MS immunocapture assay using a monoclonal antibody against HNP1-3. The antibody was amino-linked to bead, and incubated with two NAF specimens with high BF1-3 peaks (NAF-C6 and NAF-C14). Original mass spectra of NAF-C6 and C14, along with spectra of the captured proteins from each of the two samples, were shown in FIG. 3. Three peptides were captured by the antibody, and showed the exact same molecular weights and expression pattern as BF1-3 (note the relative intensity of BF3 in relation to BF1 and 2 in NAF-C14).

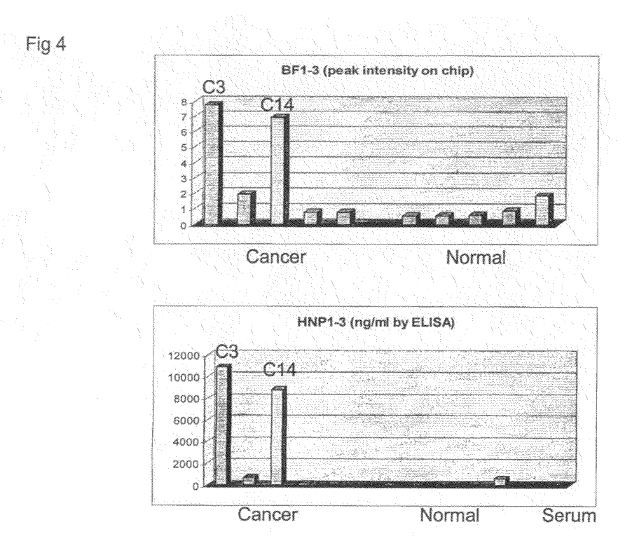

The level of HNP1-3 measured by quantative immunoassay validated the SELDI findings. Elevation of HNP1-3 in NAF C4 and C14 was further confirmed by quantitative analysis of HNP1-3 by ELISA. High peak amplitude of BF1-3 correlated with high level of HNP 1-3 measured by ELISA (FIG. 4). The concentration of HNP was 11905 ng/ml in C6, 8816 ng/ml in C14; more than 50 fold higher than the mean value of 172 ng/ml (range 19-643 ng/ml) in the normal controls.

To investigate whether elevated HNP level in breast fluid is due to contamination of blood, HNP 1-3 was also measured in a commercial pooled standard serum sample, as well as 20 banked serum samples of 4 apparently healthy women, 4 women with benign breast disease, 4 with ductal carcinoma in situ and 4 with invasive breast cancer. The level of HNP1-3 in the pooled commercial serum was determined as 41 ng/ml (value plotted in FIG. 4). HNP in banked sera ranged from 11 ng/ml to 456 ng/ml, and the mean is 44 ng/ml. The relative low concentration of HNP in serum, irrespective to cancer/non-cancer status, suggest that source of HNP in these fluid samples can not be due to contamination of blood.

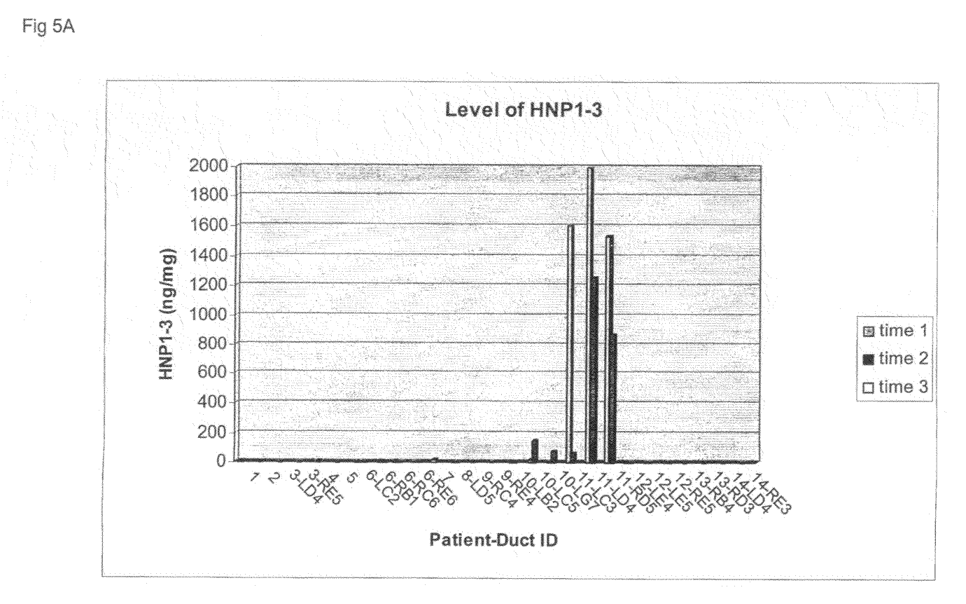

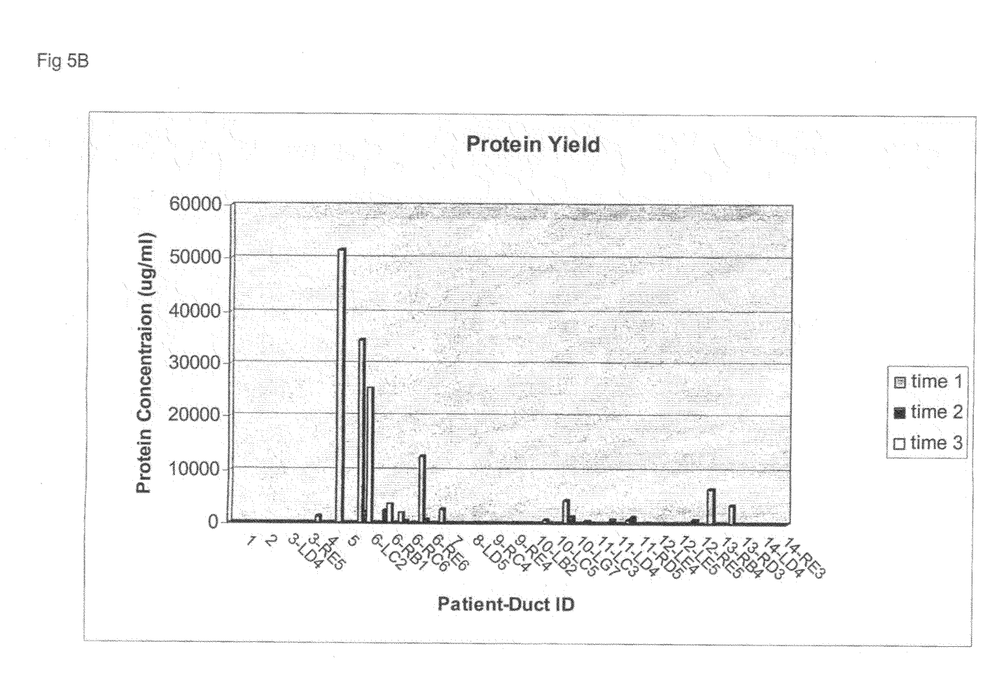

The level of HNP1-3 in DLF from women at high risk of breast cancer. Specificity of HNP1-3 to breast cancer was further tested by ELISA in 42 DLF specimens from 13 women at high risk of breast cancer (repeat lavage of both breasts at 6-12 month intervals, collected at Northwestern University). The elevation of HNP1-3 is only observed in one woman (Patient 11), whereas all 36 samples from the other 12 women tested negative (FIG. 5A). A total of six fluid samples from two duct of her left breast, one duct of her right breast, were collected at two time points 8 months apart from patient 11. High level of HNP1-3 was observed in all three ducts at the first time point and two ducts at the second time point (FIG. 5A). Based on cytology and histology data of this patient, no cancer has been detected.

To exclude the possible effect of protein yield on measurement of HNP1-3, we have also plotted the corresponding protein concentration in each sample in FIG. 5B. No correlation was observed between the level of HNP1-3 and the protein yield, low expression of HNP is not due to lack of protein in sample.

### 11.4. Example 4

NAF sample collection was completed per standard procedures. The number of samples in each category is summarized in Table 2.

TABLE 2
Bilateral NAF collection

| | Number of | |
|---|---|---|
| | Patients | Number of NAF (N) |
| Stage I/II | 28 | Paired NAF (22 × 2) |
| Invasive | | Unpaired cancerous (3) |
| Cancer | | Unpaired non-cancerous (3) |
| In-situ | 4 | Paired (2 × 2) |
| | | Unpaired cancerous(1) |
| | | Unpaired no-cancerous (1) |
| ADH | 5 | Paired (3 × 2) |
| | | Unpaired-ADH (1) |
| | | Unpaired-ADH control (1) |
| Controls | 33 | Paired NAF (20 × 2) |
| | | Unpaired (13) |
| Total | 70 | 117 |

Ductal lavage specimens were obtained from Northwestern University. This cohort consists of 149 bilateral ductal lavage specimens collected from 58 women at high risk of breast cancer (5-year Gail risk>1.6 or history of previous lobular carcinoma).

11.4.1. Proteomic Profiling

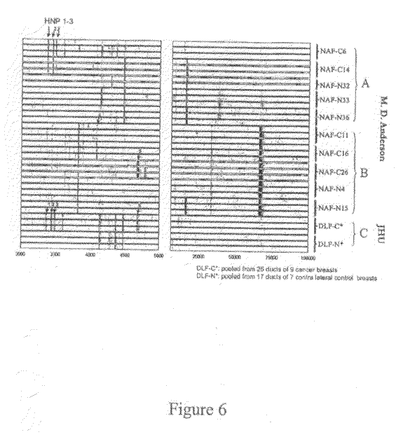

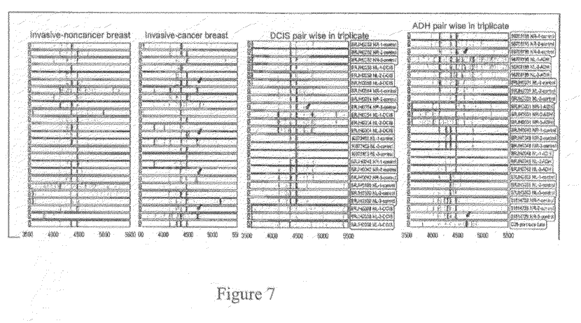

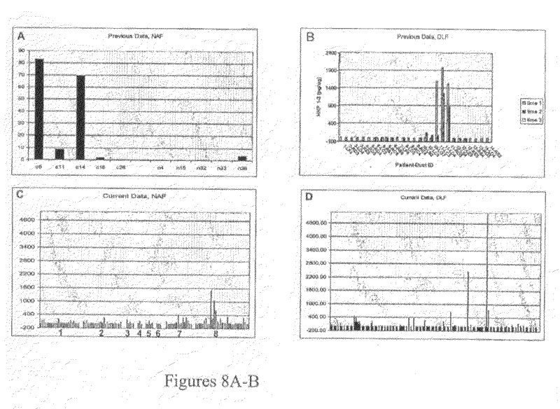

Proteomic profiling of all NAF samples in triplicate on protein chip arrays was preformed. Two previously identified potential biomarkers (HNP1-3 and BF5, FIG. 6) were detected in the current data, and their expression levels were evaluated by peak intensity (BF-5, FIG. 7) and ELISA (HNP1-3, FIG. 8), respectively.

11.4.2. Expression of HNP1-3

Elevated expression of HNP1-3 was observed in high risk population, at a much more significant level as compared to previous observation made on cancer cases.

11.4.3. Identification of BF5

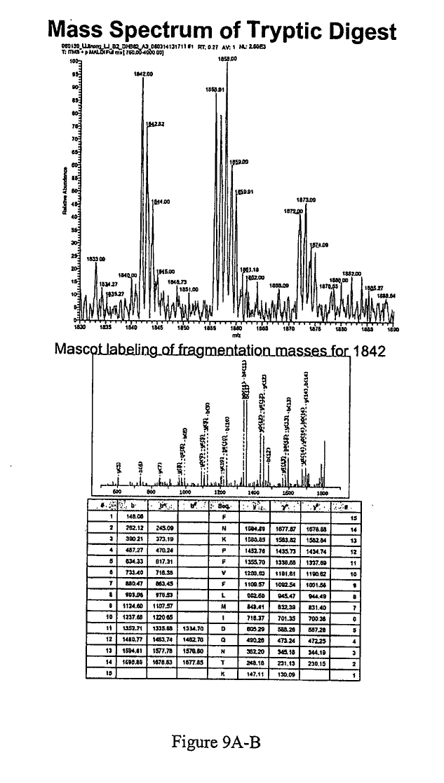

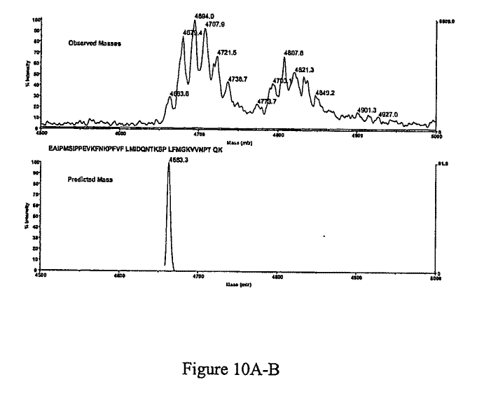

A single gel band that contained BF5 was isolated. Material eluted from the gel was sent to Johns Hopkins Core Facility for peptide finger printing and peptide sequencing. BF5 was identified as C-terminal fragments of α-1-antitrypsin inhibitor (AAT). The single band contained two C-terminal fragments designated BF-5-1 and BF-5-2. BF-5-1 was determined be a C-terminal fragment of α-1-antitrypsin inhibitor (AAT), with amino-acid sequence LEAIPMSIPPE VKFNKPFVFL MIDQNTKSPL FMGKVVNPTQ K. BF-5-2 was determined be a C-terminal fragment of α-1-antitrypsin inhibitor (AAT), with amino-acid sequence EAIPMSIPPE VKFNKPFVFL MIDQNTKSPL FMGKVVNPTQ K. Mass spectrum of tryptic digest, and Mascot fragmentation pattern for tryptic fragment 1842 (underlined), as well as comparison of observed and predicted mass of BF5-1 and 2 are shown. The multiple peaks at 4679.4, 4694.0, 4707.9 correspond to 1, 2, and 3 oxidized Methionines (M) in the parent ion 4663.6 (see FIGS. 9 and 10).

Additional specimens for validation of the markers were collected. Bilateral NAF collection from women with invasive breast cancer, DCIS, ADH, and from women that are currently free of cancer were compared. Additional DLF specimens from women at high risk of breast cancer were obtained from Northwestern University. Two previously identified biomarkers were evaluated. Elevation of the BF5s was observed in the cancer/disease breast of 4 subjects with invasive cancer (4/25), 1 subject with DCIS (1/3), and 1 subject with ADH (1/3). None of the contra-lateral control breasts showed positive for BF5, including breasts of all control subjects. BF5 appears specific (with exception in 1 subject with ADH), with limited sensitivity. HNP1-3 was observed in high risk population, at a much higher level as compared to that observed on cancer cases.

1. A method for qualifying breast cancer status in a subject comprising:
(a) measuring at least one biomarker in a biological sample from the subject, wherein the at least one biomarker is selected from the group consisting of the biomarkers of Table 1; and
(b) correlating the measurement with breast cancer status.

2. The method of claim 1, wherein the at least one biomarker is measured by capturing the biomarker on an adsorbent surface of a SELDI probe and detecting the captured biomarkers by laser desorption-ionization mass spectrometry.

3. The method of claim 1, wherein the at least one biomarker is measured by immunoassay.

4. The method of claim 1, wherein the sample is nipple aspiration fluid (NAF).

5. The method of claim 1, wherein the sample is ductal lavage fluid (DLF).

6. The method of claim 1, wherein the correlating is performed by a software classification algorithm.

7. The method of claim 1, wherein breast cancer status is selected from breast cancer and non-breast cancer.

8. The method of claim 7, wherein, if the measurement correlates with breast cancer, the method further comprises administering at least one treatment to the subject selected from the group consisting of surgery, radiation, and chemotherapy.

9. The method of claim 1, wherein breast cancer status is selected from stage I or II primary in situ breast cancer and non-breast cancer.

10. The method of claim 1, wherein the at least one biomarker is α-defensin.

11. The method of claim 10, wherein the α-defensin is α-defensin-1, α-defensin-2, or α-defensin-3

12. The method of claim 1, wherein at least two biomarkers are measured.

13. The method of claim 12, wherein the at least two biomarkers are selected from the group consisting of α-defensin-1, α-defensin-2, α-defensin-3, C-terminal fragment 1 of α-1-antitrypsin inhibitor, and C-terminal fragment 2 of α-1-antitrypsin inhibitor.

14. The method of claim 1, wherein at least three biomarkers are measured.

15. The method of claim 14, wherein the at least three biomarkers are α-defensin-1, α-defensin-2, and α-defensin-3.

16. The method of claim 1 further comprising measuring at least one biomarker, wherein the biomarker is capable of differentiating between breast cancer and non-cancer, and wherein the biomarker is not α-defensin-1, α-defensin-2, α-defensin-3, BF-4, C-terminal fragment 1 of α-1-antitrypsin inhibitor, or the C-terminal fragment 2 of α-1-antitrypsin inhibitor.

17-19. (canceled)

20. A method for determining the course of breast cancer comprising:
(a) measuring, at a first time, at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a biological sample from the subject;
(b) measuring, at a second time, the at least one biomarker in a biological sample from the subject; and
(c) comparing the first measurement and the second measurement; wherein the comparative measurements determine the course of the breast cancer.

21. (canceled)

22. A method comprising measuring at least one biomarker selected from the group consisting of α-defensin-1, α-defensin-2, α-defensin-3, BF-4, C-terminal fragment 1 of α-1-antitrypsin inhibitor and C-terminal fragment 2 of α-1-antitrypsin inhibitor in a sample from a subject.

23. A composition comprising a purified biomarker, wherein the biomarker is selected from the group consisting of α-defensin-1, α-defensin-2, α-defensin-3, BF-4, C-terminal fragment 1 of α-1-antitrypsin inhibitor, and C-terminal fragment 2 of α-1-antitrypsin inhibitor.

24-25. (canceled)

26. A kit comprising:
(a) a solid support comprising at least one capture reagent attached thereto, wherein the capture reagent binds at least one biomarker selected from the group consisting of α-defensin-1, α-defensin-2, α-defensin-3, C-terminal fragment 1 of α-1-antitrypsin inhibitor, and C-terminal fragment 2 of α-1-antitrypsin inhibitor; and
(b) instructions for using the solid support to detect the at least one biomarker.

27-33. (canceled)

34. A software product comprising:
(a) code that accesses data attributed to a sample, the data comprising measurement of at least one biomarker selected from the group consisting of α-defensin-1, α-defensin-2, and α-defensin-3, BF-4, C-terminal fragment 1 of α-1-antitrypsin inhibitor, and C-terminal fragment 2 of α-1-antitrypsin inhibitor; and
(b) code that executes a classification algorithm that classifies the breast cancer status of the sample as a function of the measurement.

35-37. (canceled)

38. A method for identifying a compound that interacts with a biomarker selected from the group consisting of α-defensin-1, α-defensin-2, and α-defensin-3, BF-4, C-terminal fragment 1 of α-1-antitrypsin inhibitor, and C-terminal fragment 2 of α-1-antitrypsin inhibitor, wherein said method comprises:
(a) contacting the biomarker with a test compound; and
(b) determining whether the test compound interacts with the biomarker.

39. (canceled)

40. A method of treating a condition in a subject, wherein said method comprises:
administering to a subject a therapeutically effective amount of a compound, wherein said compound modulates the expression or post-translational processing of a biomarker selected from the group consisting of α-defensin-1, α-defensin-2, α-defensin-3, BF-4, C-terminal fragment 1 of α-1-antitrypsin inhibitor, and C-terminal fragment 2 of α-1-antitrypsin inhibitor.

41. The method of claim 40 wherein said condition is breast cancer.
